(19) United States
(12) Patent Application Publication
(10) Pub. No.: US 2025/0302750 A1
(43) Pub. Date: Oct. 2, 2025

(54) PHARMACEUTICAL COMPOSITION OF BEMPEDOIC ACID

(71) Applicant: Renata Pharmaceuticals (Ireland) Ltd, Dublin (IE)

(72) Inventors: Syed S. Kaiser Kabir, Dhaka (BD); Syed Omar Kabir, Dhaka (BD); Ganesh Vinayak Gat, Dhaka (BD); Rambabu Boorugu, Dhaka (BD); Koushik Esh, Dhaka (BD)

(21) Appl. No.: 18/864,027

(22) PCT Filed: May 8, 2023
(86) PCT No.: PCT/EP2023/062107

§ 371 Date: Nov. 8, 2024

Related U.S. Application Data
(60) Provisional application No. 63/339,743, filed on May 9, 2022.

(51) Int. Cl. A61K 9/20; A61K 31/20

(52) U.S. Cl. CPC A61K 9/2018; A61K 9/2054; A61K 9/2059; A61K 9/2077; A61K 9/2095; A61K 31/20

(57) ABSTRACT

Disclosed herein is an oral pharmaceutical composition of Bempedoic acid and process for preparing the same, wherein said oral pharmaceutical composition comprising therapeutically effective amount of Bempedoic acid and pharmaceutically acceptable adjuvants, and wherein the composition is tablet.

### PRIORITY CLAIM

This Application claims the benefit of U.S. Provisional Application No. 63/339,743 filed May 9, 2022, which application is incorporated herein by reference.

### FIELD OF THE INVENTION

The present invention relates to a field of an oral pharmaceutical compositions in general, and in particular to pharmaceutical composition of Bempedoic acid and a process for preparing the same.

### BACKGROUND OF THE INVENTION

Bempedoic acid (ETC-1002) is an oral, first-in class, small molecule designed to lower low-density lipoprotein cholesterol (LDL-C). ETC-1002 is an agent that has been shown to lower low-density lipoprotein cholesterol (LDL-C) by direct inhibition of hepatic adenosine triphosphate citrate lyase, leading to reduced de novo cholesterol synthesis and increased LDL receptor expression. Bempedoic acid (ETC-1002) is a small molecule that inhibits adenosine triphosphate-citrate lyase (ACL), an enzyme upstream of 3-hydroxy-3-methylglutaryl coenzyme A (HMG-COA) reductase in the cholesterol biosynthesis pathway. Bempedoic acid, like both statins and Ezetimibe, up-regulates LDL-C receptors.

Bempedoic acid (ETC-1002) is in the drug product BCS Class II compound. It is poorly soluble in water and highly permeable. US Patent application no. U.S. Pat. No. 20,180,338922, discloses that, Bempedoic acid in the solid state, exhibits poor flow characteristics and is very sticky. Its stickiness adversely impacts various stages during development of pharmaceutical formulations including weighing, blending, granulation and compression. These problems adversely impact drug manufacturing operations, notably tablet compression (low rpm operation, weight variation, frequent machine stoppage; etc.). It further discloses that, standard granulation of Bempedoic acid only marginally reduces the sticky behavior thereby improving processability. Bempedoic acid also has a relatively low melting point, 88-91° C., and as such contributes to the diminished plasticity of the bulk. To overcome said problem said prior art suggested addition of lubricant intragranularly i.e. during dry mixing of API and excipients with lengthy mixing time.

After rigorous experimentation it was surprisingly found that oral compositions comprising Bempedoic acid, in solid oral tablet form provide the desired stability profile. The said composition is cost effective, safe and process of obtaining it is less complex. The present inventors have also surprisingly found that Bempedoic acid tablet without intragranular lubricant can be produce, while maintaining all tablet and tableting parameters.

### SUMMARY OF THE INVENTION

In accordance with the principal aspect of the present invention, there is provided oral immediate release pharmaceutical composition comprising Bempedoic acid that exhibits tablet composition.

In a first aspect, the invention relates to a high-loaded pharmaceutical granulate comprising Bempedoic acid, at least one hydrophilic binder, at least one wetting agent and at least one disintegrant. In an embodiment, the granulate comprises Bempedoic acid, one wetting agent, one hydrophilic binder and one hydrophilic disintegrant.

In a another aspect, the invention relates to a process for making said pharmaceutical granulate comprising the steps of i) Preparing the granulation liquid comprising a solution of a wetting agent in a solvent comprising water; ii) Charging granulation equipment with Bempedoic acid, at least one hydrophilic binder, at least one hydrophilic disintegrant and, optionally, with other excipients; iii) Granulating the mixture using the aqueous solution from the step i) as the granulation liquid; and iv) Drying the granulate.

In another embodiment of the present invention, the wet granulation process to prepare granules includes the following steps: i) forming a powder blend of Bempedoic acid and at least one pharmaceutically acceptable excipient; ii) adding a granulation liquid to the powder blend under agitation to form a wet mass; iii) granulating the wet mass to form moist granules; iv) drying the moist granules and iv) milling the dried granules.

In one aspect, the invention relates to a process for making an immediate release tablet for oral administration of Bempedoic acid comprising the steps of i) Preparing a pharmaceutical granulate comprising Bempedoic acid, at least one hydrophilic binder, at least one wetting agent and at least one disintegrant by granulating a mixture comprising Bempedoic acid, at least one hydrophilic binder and at least one disintegrant by a granulation liquid comprising an aqueous solution of a wetting agent, and drying the wet granulate; ii) Mixing the optionally milled granulate with at least one further pharmaceutically acceptable excipient, which preferably comprises at least one lubricant; and iii) Compressing the mixture into a tablet.

In another embodiment of the present invention, the tableting process to prepare tablet includes the following steps: i) forming a powder blend of Bempedoic acid and at least one pharmaceutically acceptable excipient; ii) adding a granulation liquid to the powder blend under agitation to form a wet mass; iii) granulating the wet mass to form moist granules; iv) drying the moist granules; v) milling the dried granules; vi) Lubricating the dried granules using blender and vii) Compress the lubricated blend into tablet.

In another embodiment, the present disclosure provides for pharmaceutical composition comprising: Bempedoic acid dry blend free of lubricant. Non limiting examples of lubricant are colloidal silicon dioxide, sodium stearyl fumarate, and magnesium stearate; and a pharmaceutically acceptable excipient.

In particular, the unit dosage forms, particularly the tablets, in accordance with the present invention are characterized by a dissolution rate of more than 70%, of the dose of Bempedoic acid in 30 minutes when tested by USP dissolution test in phosphate buffer pH6.6, in 900 ml standard vessel, with paddle apparatus 2 of paddle speed 50 rpm.

In an alternative embodiment, a comminuting mill may be used in lieu of the screen or sieve. Examples of a comminuting mill include, but are not limited to, a Stokes oscillator, a Colton rotary granulator, a Fitzpatrick comminuting mill, a Stokes tornado mill.

In yet another alternative embodiment, a high-speed mixer equipped with, for example a chopper blade, may be used to replace either the screen or the comminuting mill. In this case, the granulating step is called kneading. This, for example, allows the wet massing and granulating to be combined into a single step.

Sticking problems were observed with the tablet of Bempedoic acid during tableting process. Surprisingly it was found that dried granules having LOD (Loss on drying) around 0.5% to 2% at 70° C. do not have such sticking problems. Hence, in one embodiment, the present invention relates to tablet as described herein containing dried granules having LOD in an amount of more than about 0.5% at 70° C.

In another embodiment, the dried granulate comprises less than 2% or less than 1.5%, of LOD.

In yet another embodiment, the present disclosure provides for a pharmaceutical composition wherein the composition comprises at least 40% and nor more than 95% Bempedoic acid by weight of the total composition.

In some embodiments, the present disclosure provides for a pharmaceutical composition wherein the composition further comprises one or more of: magnesium stearate, hydroxypropyl cellulose, a saccharide, microcrystalline cellulose and a starch.

In some aspects, the present disclosure provides for a pharmaceutical composition wherein the saccharide, when present, is lactose monohydrate.

In some aspects, the present disclosure provides for a pharmaceutical composition wherein the amount of magnesium stearate is between 1 mg and 10 mg, the amount of hydroxypropyl cellulose (HPC) is between 5 mg and 25 mg, the amount of saccharide is between 15 mg and 100 mg, the amount of microcrystalline cellulose is between 50 mg and 150 mg and the amount of sodium starch glycolate is between 5 mg and 50 mg.

In some aspects, the present disclosure provides for a pharmaceutical composition wherein the amount of Bempedoic acid is between 80 mg and 250 mg. In some aspects, the amount of Bempedoic acid is between 100 mg and 200 mg. In some aspects, the amount of Bempedoic acid is between 150 mg and 200 mg.

In some aspects, the present disclosure provides for a pharmaceutical composition wherein the amount of Bempedoic acid is 180 mg.

In some aspects, the present disclosure provides for a pharmaceutical composition comprising Bempedoic acid as described herein that has improved flowability characteristics as described herein.

In some aspects, the present disclosure provides for a pharmaceutical composition comprising Bempedoic acid as described herein that has improved non-stickiness characteristics as described herein.

In some aspects, the present disclosure provides for a pharmaceutical composition comprising Bempedoic acid as described herein that has improved chemo-physical characteristics such as particle size, surface area, pore volume, flow property of granules and other properties as described herein.

In another aspect, the present invention relates to immediate release oral tablet comprising Bempedoic acid and one or more pharmaceutical acceptable excipient, wherein lubricant present specifically in external phase of the tablet.

The details of one or more embodiments of the inventions are set forth in the description below. Others features, objects and advantages of the inventions will be apparent from the description and claims.

### DETAILED DESCRIPTION OF THE INVENTION

Terms used in the claims and specification are defined as set forth below unless otherwise specified. Further, if any term or symbol used herein is not defined as set forth below, it shall have its ordinary meaning in the art.

The practice of the present invention includes the use of conventional techniques of organic chemistry, molecular biology (including recombinant techniques), microbiology, cell biology, biochemistry, and immunology, which are within the skill of the art.

As used herein and in the appended claims, singular articles such as “a,” “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, including the upper and lower bounds of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein the term “pharmaceutical composition” means, for example, a mixture containing a specified amount of a therapeutic compound, e.g. a therapeutically effective amount, of a therapeutic compound in a pharmaceutically acceptable carrier to be administered to a mammal, e.g., a human in order to treat kinase dependent diseases.

As used herein the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

The concentration of therapeutic compound in the pharmaceutical composition is present in an amount, e.g. in a therapeutically effective amount, which will depend on absorption, inactivation and excretion rates of the drug as well as other factors known to one of ordinary skill in the art. Furthermore, it is to be noted that dosage values will also vary with the severity of the condition to be alleviated. It is to be further understood that for any particular recipient, specific dosage regimens should be adjusted over time according to the individual need and the professional judgment of the person administering or supervising the administration of the pharmaceutical compositions. The therapeutic compound may be administered once or may be divided into a number of smaller doses to be administered at varying intervals of time. Thus, an appropriate amount, e.g. an appropriate therapeutically effective amount, is known to one of ordinary skill in the art.

For example, the dose of the therapeutic compound will be in the range from about 0.1 to about 100 mg per kilogram body weight of the recipient per day. Alternatively lower doses may be given, for example doses of 0.1 to 200 mg; 0.1 to 50 mg; or 0.1 to 20 mg per kilogram body weight per day. The effective dosage range of the pharmaceutically acceptable salts may be calculated based on the weight of the active moiety to be delivered. If the salt exhibits activity itself, the effective dosage may be estimated as above using the weight of the salt, or by other means known to those skilled in the art.

The term “sufficient amount” means an amount sufficient to produce a desired effect, e.g., an amount sufficient to modulate protein aggregation in a cell.

The term “administering” or “administration” of a drug and/or therapy to a subject (and grammatical equivalents of this phrase) refers to both direct or indirect administration, which may be administration to a subject by a medical professional, may be self-administration, and/or indirect administration, which may be the act of prescribing or inducing one to prescribe a drug and/or therapy to a subject.

Unless defined otherwise, all technical and scientific terms used herein have the meaning commonly understood by a person skilled in the art to which this invention belongs.

As used herein the term “immediate-release” refers to the rapid release of the majority of the therapeutic compound, e.g., greater than about 50%, about 55%, about 60%, about 65%, about 70%, about 75%, about 80%, or about 90% within a relatively short time, e.g., within 1 hour, 40 minutes, 30 minutes or 20 minutes after oral ingestion. Particularly useful conditions for immediate-release are release of at least or equal to about 80% of the therapeutic compound within thirty minutes after oral ingestion. The particular immediate-release conditions for a specific therapeutic compound will be recognized or known by one of ordinary skill in the art.

As used herein the term “excipient” refers to a pharmaceutically acceptable ingredient that is commonly used in the pharmaceutical technology for preparing granule and/or solid oral dosage formulations. Examples of categories of excipients include, but are not limited to, binders, disintegrants, lubricants, glidants, stabilizers, fillers and diluents. One of ordinary skill in the art may select one or more of the aforementioned excipients with respect to the particular desired properties of the granule and/or solid oral dosage form by routine experimentation and without any undue burden. The amount of each excipient used may vary within ranges conventional in the art.

As used herein, the term “wet granulation” refers to the general process of using a granulation liquid in the granulation process to subsequently form granules, as discussed in Remington: The Science and Practice of Pharmacy, 20^{th} Edition (2000), Chapter 45, which is hereby incorporated by reference.

Unless otherwise specified, tap density is measured by the USP bulk density and tapped density test <616> and LOD is measured by the USP LOD test <731>.

In accordance with the principal aspect of the present invention, there is provided oral immediate release pharmaceutical composition comprising Bempedoic acid that exhibits tablet composition.

In a first aspect, the invention relates to a high-loaded pharmaceutical granulate comprising Bempedoic acid, at least one hydrophilic binder, at least one wetting agent and at least one disintegrant. In an embodiment, the granulate comprises Bempedoic acid, one wetting agent, one hydrophilic binder and one hydrophilic disintegrant.

In a another aspect, the invention relates to a process for making said pharmaceutical granulate comprising the steps of i) Preparing the granulation liquid comprising a solution of a wetting agent in a solvent comprising water; ii) Charging granulation equipment with Bempedoic acid, at least one hydrophilic binder, at least one hydrophilic disintegrant and, optionally, with other excipients; iii) Granulating the mixture using the aqueous solution from the step i) as the granulation liquid; and iv) Drying the granulate.

In another embodiment of the present invention, the wet granulation process to prepare granules includes the following steps: i) forming a powder blend of Bempedoic acid and at least one pharmaceutically acceptable excipient; ii) adding a granulation liquid to the powder blend under agitation to form a wet mass; iii) granulating the wet mass to form moist granules; iv) drying the moist granules and iv) milling the dried granules.

In one aspect, the invention relates to a process for making an immediate release tablet for oral administration of Bempedoic acid comprising the steps of i) Preparing a pharmaceutical granulate comprising Bempedoic acid, at least one hydrophilic binder, at least one wetting agent and at least one disintegrant by granulating a mixture comprising Bempedoic acid, at least one hydrophilic binder and at least one disintegrant by a granulation liquid comprising an aqueous solution of a wetting agent, and drying the wet granulate; ii) Mixing the optionally milled granulate with at least one further pharmaceutically acceptable excipient, which preferably comprises at least one lubricant; and iii) Compressing the mixture into a tablet.

In another embodiment of the present invention, the tableting process to prepare tablet includes the following steps: i) forming a powder blend of Bempedoic acid and at least one pharmaceutically acceptable excipient; ii) adding a granulation liquid to the powder blend under agitation to form a wet mass; iii) granulating the wet mass to form moist granules; iv) drying the moist granules; v) milling the dried granules; vi) Lubricating the dried granules using blender and vii) Compress the lubricated blend into tablet.

In another embodiment, the present disclosure provides for pharmaceutical composition comprising: Bempedoic acid dry blend free of lubricant. Non limiting examples of lubricant are colloidal silicon dioxide, sodium stearyl fumarate, and magnesium stearate; and a pharmaceutically acceptable excipient.

In particular, the unit dosage forms, particularly the tablets, in accordance with the present invention are characterized by a dissolution rate of more than 70%, of the dose of Bempedoic acid in 30 minutes when tested by USP dissolution test in phosphate buffer pH6.6, in 900 ml standard vessel, with paddle apparatus 2 of paddle speed 50 rpm.

In an alternative embodiment, a comminuting mill may be used in lieu of the screen or sieve. Examples of a comminuting mill include, but are not limited to, a Stokes oscillator, a Colton rotary granulator, a Fitzpatrick comminuting mill, a Stokes tornado mill.

In yet another alternative embodiment, a high-speed mixer equipped with, for example a chopper blade, may be used to replace either the screen or the comminuting mill. In this case, the granulating step is called kneading. This, for example, allows the wet massing and granulating to be combined into a single step.

Sticking problems were observed with the tablet of Bempedoic acid during tableting process. Surprisingly it was found that dried granules having LOD (Loss on drying) around 0.5% to 2% at 70° C. do not have such sticking problems. Hence, in one embodiment, the present invention relates to tablet as described herein containing dried granules having LOD in an amount of more than about 0.5% at 70° C.

In another embodiment, the dried granulate comprises less than 2% or less than 1.5%, of LOD.

In yet another embodiment, the present disclosure provides for a pharmaceutical composition wherein the composition comprises at least 40% and nor more than 95% Bempedoic acid by weight of the total composition.

In some embodiments, the present disclosure provides for a pharmaceutical composition wherein the composition further comprises one or more of: magnesium stearate, hydroxypropyl cellulose, a saccharide, microcrystalline cellulose and a starch.

In some aspects, the present disclosure provides for a pharmaceutical composition wherein the saccharide, when present, is lactose monohydrate.

In some aspects, the present disclosure provides for a pharmaceutical composition wherein the amount of magnesium stearate is between 1 mg and 10 mg, the amount of hydroxypropyl cellulose (HPC) is between 5 mg and 25 mg, the amount of saccharide is between 15 mg and 100 mg, the amount of microcrystalline cellulose is between 50 mg and 150 mg and the amount of sodium starch glycolate is between 5 mg and 50 mg.

In some aspects, the present disclosure provides for a pharmaceutical composition wherein the amount of Bempedoic acid is between 80 mg and 250 mg. In some aspects, the amount of Bempedoic acid is between 100 mg and 200 mg. In some aspects, the amount of Bempedoic acid is between 150 mg and 200 mg.

In some aspects, the present disclosure provides for a pharmaceutical composition wherein the amount of Bempedoic acid is 180 mg.

In some aspects, the present disclosure provides for a pharmaceutical composition comprising Bempedoic acid as described herein that has improved flowability characteristics as described herein.

In some aspects, the present disclosure provides for a pharmaceutical composition comprising Bempedoic acid as described herein that has improved non-stickiness characteristics as described herein.

In some aspects, the present disclosure provides for a pharmaceutical composition comprising Bempedoic acid as described herein that has improved chemo-physical characteristics such as particle size, surface area, pore volume, flow property of granules and other properties as described herein.

In another aspect, the present invention relates to immediate release oral tablet comprising Bempedoic acid and one or more pharmaceutical acceptable excipient, wherein lubricant present specifically in external phase of the tablet.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in a concentration from about 0 to about 50% by weight of the composition (e.g., by the tablet weight).

Examples of pharmaceutically acceptable binders include, but are not limited to, starches; celluloses and derivatives thereof, for example, microcrystalline cellulose, e.g., AVICEL PH from Mingtai (Taiwan), hydroxypropyl cellulose hydroxylethyl cellulose and hydroxylpropylmethyl cellulose, e.g. METHOCEL from Arhland; sucrose; dextrose; corn syrup; polysaccharides; povidone and gelatin. The binder may be present in a concentration from about 0 to about 50% by weight of the composition (e.g., by the tablet weight).

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, and sucrose. The filler may be present in a concentration from about 0 to about 80% by weight of the composition (e.g., by the tablet weight).

The next step is wet massing the powder blend by adding a granulation liquid while agitating the powder blend until the powder blend is wetted with the granulation liquid to form a wet mass. For example, 10% to 60% (w/w) granulation liquid is added to the powder blend. Alternatively, 10% to 15% (w/w) granulation liquid may be added to the powder blend. The granulation liquid, for example is pharmaceutically acceptable and volatile. Examples of suitable granulation liquids include, but are not limited to, water (e.g. purified water), organic solvents (e.g., methanol, ethanol, isopropanol, acetone) either alone or in combination. An example of a combination granulation liquid includes water, ethanol and isopropanol together.

Alternatively, the wet granulation process may begin with the therapeutic compound as a powder by itself.

During wet massing, the granulation liquid that is introduced to the powder is a solvent containing one or several dissolved excipients, e.g. a binder and/or a surfactant. Irrespective of how wet-massing takes place, after wet-massing, the powder blend is wetted by the granulation liquid. In one exemplary embodiment, purified water is used as the granulation liquid.

Subsequently after processing with the granulation liquid, the wet mass may be optionally sieved forming moist, or damp, granules. The wet mass, for example, may be sieved through a mesh, such as a 1 to up to 7 mm, e.g. 4- or 8-mesh screen. One of ordinary skill in the art may select the appropriate size of the screen in order to form the most appropriate granule size.

The moist granules, for example, are subsequently dried. For example, the moist granules may be collected on trays and transferred to a drying oven. Alternatively, the moist granules may be placed in a drying cabinet with circulating air current and thermostatic heat control. Yet another option is to dry the moist granules in a fluid-bed drier. In this exemplary embodiment, the moist granules are suspended and agitated in a warm air stream such that the moist granules are maintained in motion. For example, the air temperature may be from about room temperature to about 90° C. The moist granules are dried to a loss on drying (“LOD”) value less than or equal to about five percent, e.g., less than two percent, e.g., 0.5% to 1.8%, by weight of the composition.

Yet another option is a single pot process with granulation and drying in the same equipment (for example, a high shear mixer with a double wall for drying like a Zanchetta Roto P or Turbosphere Moritz).

Drying may take place within or apart from the pharmaceutical granulation equipment.

Subsequent to drying, the granule may be further sieved, i.e., dry screened, alone or in combination with at least one excipient. This typically results in a more uniform particle size of granules, preparing the granules for further processing into a solid oral dosage form.

The granules may be formulated with additional pharmaceutically acceptable excipients to form an intimate mixture that is subsequently formed into an oral form, e.g., solid oral dosage forms, such as tablets, pills, lozenges, caplets, capsules or sachets. As used herein, the term “external phase” refers to the additional excipients that are added to the granules prior to forming the final dosage form. Any additional excipients used may be sieved separately from the granules or concurrently with the sieving of the granules as described in the aforementioned dry sieving step. One of ordinary skill in the art will appreciate the necessary particle size of each component that is necessary for the particular pharmaceutical composition being formulated. For example, suitable particle sizes, include those of less than equal to 1,000 μm, 750 μm, 500 μm or 250 μm. Assembling of the granules with the external phase into an intimate mixture may be accomplished using any conventional pharmaceutical process as known by one of ordinary skill in the art, for example, blending, compressing, co-milling, compacting, or co-micronizing.

The blended mixture may be subsequently compacted into a tablet (e.g., by using a tablet press).

A commonly used pharmaceutically acceptable excipient to add in the external phase is a glidant. Such an excipient facilitates the flow of the blended mixture in the processing equipment.

Examples of pharmaceutically acceptable glidants include, but are not limited to, colloidal silica, magnesium trisilicate, starches, talc, tribasic calcium phosphate, aluminum stearate, magnesium carbonate, magnesium oxide and powdered cellulose. The glidant may be present in a concentration from about 0.1 to 10%, e.g. from 0.1 to 10% e.g. 1.5%, by weight of the total weight of the pharmaceutical composition.

Another commonly used pharmaceutically acceptable excipient to add to the external phase is a lubricant. Such an excipient helps to avoid any sticking in the processing equipment. Although a lubricant enhances processability, it may impact the release of the therapeutic compound from the dosage form. Often, a lubricant is hydrophobic and consequently retards or slows down the release of a therapeutic compound in an immediate release dosage form. This reduction of lubricant concentration results in a pharmaceutical composition with a better dissolution profile than if no surfactant is used. Without being bound to any particular theory, the use of a lubricant may prevent access of water to the other excipients due to its hydrophobicity, and consequently slow down solubilization. For example, in exemplary embodiments of the present invention, the concentration of the lubricant is less than 10% by weight of the pharmaceutical composition.

Examples of lubricants, e.g. pharmaceutically acceptable lubricants include, but are not limited to, talc, magnesium stearate, aluminum stearate, calcium stearate, magnesium carbonate, polyethylene glycol, glyceryl behenate, stearic acid, hydrogenated castoril, glyceryl monostearate and sodium stearyl fumarate. The lubricant may be present in a concentration form about 0 to 10%, e.g. 0 to 10%, alternatively about 5%, e.g. 5%, by weight of the total weight of the pharmaceutical composition.

The tablets of present invention may be coated, e.g., by a film coat, for better handling and cosmetic purposes. The film coating may contain pharmaceutically acceptable colourants or flavouring agents. This film coating should be rapidly dissolvable in the stomach environment (a non-enteric coating) to minimize the latent period prior to release and should not have any other influence on the release characteristics of the active pharmaceutical ingredient.

A plurality of tablets may be packed in a suitable package material, which advantageously protects them against light and moisture; blisters or bottles made from aluminum and/or hard polymer (i.e. HDPE/PVC/PE/PVDC or PVC/PVDC) are examples of such package materials. Mutatis mutandis, the pharmaceutical granulate of the present invention and at least one further suitable excipient may be used for making pharmaceutical capsules or sachets. Such dosage forms typically exhibit the same dissolution properties and may comprise the same dose amounts of the active substance as those of the above tablets.

The following examples are illustrative, but do not serve to limit the scope of the invention described herein. The examples are meant only to suggest a method of practicing the present invention.

Quantities of ingredients, represented by percentage by weight of the pharmaceutical composition, used in each example are set forth in the respective tables located after the respective descriptions.

### Example 1-5:

TABLE NO. 1

| | Example | Example | Example | Example | Example | Example | Example |
|---|---|---|---|---|---|---|---|
| | 1 | 2 | 3 | 4 | 5 | 6 | 7 |
| Ingredients | mg/tablet | mg/tablet | mg/tablet | mg/tablet | mg/tablet | mg/tablet | mg/tablet |
| Bempedoic Acid | 180.000 | 180.000 | 180.000 | 180.000 | 180.000 | 180.000 | 180.000 |
| Microcrystalline | 87.250 | 87.250 | 87.250 | 87.250 | 106.750 | 87.250 | 87.250 |
| Cellulose | | | | | | | |
| Lactose | 28.500 | 28.500 | 28.500 | 28.500 | 5.750 | 28.500 | 28.500 |
| Monohydrate | | | | | | | |
| Sodium Starch | 6.500 | 6.500 | 3.250 | 13.000 | 6.500 | 4.000 | 8.125 |
| Glycolate | | | | | | | |
| Hydroxypropyl | 6.500 | 13.000 | 6.500 | 6.500 | 9.750 | 9.750 | 3.250 |
| Cellulose | | | | | | | |
| Sodium Starch | 6.500 | — | 9.750 | — | 6.500 | 4.000 | 8.125 |
| Glycolate | | | | | | | |
| Colloidal Silicon | 4.875 | 4.875 | 4.875 | 4.875 | 4.875 | 3.250 | 3.250 |
| Dioxide | | | | | | | |
| Magnesium | 4.875 | 4.875 | 4.875 | 4.875 | 4.875 | 9.750 | 6.500 |
| Stearate | | | | | | | |
| Opadry II | 9.750 | 9.750 | 9.750 | 9.750 | 9.750 | 9.750 | 9.750 |

### Process:

Granulation Process;
1) Dissolve Hydroxypropyl cellulose in purified water under stirring
2) Mix the weighed amount of Bempedoic Acid, Microcrystalline cellulose, Lactose monohydrate, Sodium starch glycolate in bin blender and sieve the mixture by sieve #40.
3) Mix the components from the step 2 in a High Shear Mixer (HSM) granulator and granulate the mixture by adding the solution from the step 1.
4) Sieve the granulated product from the step 3 through a #05 mesh.
5) Dry the wet granulate from step 4 in a fluid bed drier until a LOD <2% at 70° C.
6) Mill the dried granulate from step 5 through a #12 mesh screen by using a Co-Mill and sieve using #24mesh screen.

Tabletting Process;
7) Sift Sodium Starch Glycolate (Example 1, Example 3, Example 5, Example 6 and Example 7), Colloidal silicon dioxide and Magnesium Stearate through #40 mesh screen.
8) Mix the granulate from step 6 with components from step 7 in blender.
9) Weight magnesium stearate and mix it with the product from step 8.
10) Compress the final blend from the step 9 in a tablet press into a tablet.

Coating Process;
11) Weight the Opadry II and stir it for 45 minutes in purified water.
12) Warm-up tablets from step 10 up to a tablet temperature of 45° C. in a coater.
13) Coat the tablets with the suspension from step 11 up to a weight gain of 3%. Tablets temperature should be between 38-43° C.
14) Dry the coated tablets.

### Observations

In-process parameter of dried granules and lubricated blend are checked as per USP Methods. The LOD of several lots of dried granules were determined by USP LOD test <731> and are shown in Table 2 below. LOD were determined using Mettler Toledo's Moisture Analyzer. 1gm of dried granules were heated at 70° C., until constant weight had been achieved. The tap density of several lots of lubricated granules were determined by USP bulk density and tapped density test <616> and are shown in Table 2 below.

TABLE NO. 2

| | | | | Exam- | Exam- | Exam- | Exam- |
|---|---|---|---|---|---|---|---|
| Parameters | Example 1 | Example 2 | Example 3 | ple 4 | ple 5 | ple 6 | ple 7 |
| LOD (%) | 2.09 | 2.21 | 1.74 | 1.83 | 1.63 | 0.85 | 1.16 |
| BD (g/ml) | 0.48 | 0.46 | 0.46 | 0.41 | 0.47 | 0.43 | 0.44 |
| TD (g/ml) | 0.59 | 0.54 | 0.59 | 0.50 | 0.58 | 0.53 | 0.57 |
| Carr's index (%) | 22.92 | 17.39 | 28.26 | 21.95 | 23.40 | 23.26 | 29.55 |
| Hausner Ratio | 1.23 | 1.17 | 1.28 | 1.22 | 1.23 | 1.23 | 1.30 |

| | |
|---|---|
| Tablet Sticking | Tablet sticking was absent |
| defect (while | |
| Tableting) | |

1. An oral immediate release pharmaceutical composition comprising Bempedoic acid.

2. The pharmaceutical composition as claimed in claim 1 that exhibits tablet composition.

3. The pharmaceutical composition as claimed in claim 2, wherein the composition further comprises one or more of: magnesium stearate, hydroxypropyl cellulose, a saccharide, microcrystalline cellulose and a starch.

4. The pharmaceutical composition as claimed in claim 3, wherein the saccharide, is lactose monohydrate.

5. The pharmaceutical composition as claimed in claim 3, wherein Bempedoic acid is a dry blend free of lubricant.

6. The pharmaceutical composition as claimed in claim 3, wherein one or more lubricants are present in external phase of granules.

7. The pharmaceutical composition as claimed in claim 6, wherein the one or more lubricants are selected from; colloidal silicon dioxide, sodium stearyl fumarate, and magnesium stearate.

8. The pharmaceutical composition as claimed in claim 3, wherein the amount of magnesium stearate is between 1 mg and 10 mg, the amount of hydroxypropyl cellulose (HPC) is between 5 mg and 25 mg, the amount of saccharide is between 15 mg and 100 mg, the amount of microcrystalline cellulose is between 50 mg and 150 mg and the amount of sodium starch glycolate is between 5 mg and 50 mg.

9. The pharmaceutical composition as claimed in claim 3, which comprises at least one hydrophilic binder, at least one disintegrant and at least one lubricant.

10. A pharmaceutical granulate comprising Bempedoic acid, at least one hydrophilic binder, at least one disintegrant and at least one lubricant.

11. The pharmaceutical granulate as claimed in claim 10, wherein the amount of bempedoic acid is between 80 mg and 250 mg.

12. The dry granulate as claimed in claim 10, wherein LOD of dried granules less than 2% at 70° C.

13. A process for making pharmaceutical granulate comprising following steps:
i) preparing the granulation liquid comprising a solution of a wetting agent in a solvent comprising water;
ii) charging granulation equipment with Bempedoic acid, at least one hydrophilic binder, at least one hydrophilic disintegrant and, optionally, with other excipients;
iii) granulating the mixture using the aqueous solution from the step i) as the granulation liquid; and
iv) drying the granulate.

14. A wet granulation process to prepare granules includes following steps:
i) forming a powder blend of bempedoic acid and at least one pharmaceutically acceptable excipient;
ii) adding a granulation liquid to the powder blend under agitation to form a wet mass;
iii) granulating the wet mass to form moist granules iv) drying the moist granules; and
iv) milling the dried granules.

15. A process for making an immediate release tablet for oral administration of Bempedoic acid comprising following steps:
i) forming a powder blend of Bempedoic acid and at least one pharmaceutically acceptable excipient;
ii) adding a granulation liquid to the powder blend under agitation to form a wet mass;
iii) granulating the wet mass to form moist granules;
iv) drying the moist granules;
v) milling the dried granules;
vi) lubricating the dried granules using blender;
vii) compress the lubricated blend into tablet; and
viii) coating over the compressed tablets.
